(19) United States
(12) Patent Application Publication
(10) Pub. No.: US 2025/0250628 A1
(43) Pub. Date: Aug. 7, 2025

(54) METHODS OF IDENTIFYING AND/OR CHARACTERIZING A POLYNUCLEOTIDE OF INTEREST

(71) Applicant: Regeneron Pharmaceuticals, Inc., Tarrytown, NY (US)

(72) Inventors: Shuli TANG, White Plains, NY (US); Gaoyuan LIU, Glenview, IL (US); Yuetian YAN, Chappaqua, NY (US); Shunhai WANG, Scarsdale, NY (US); Ning LI, New Canaan, CT (US)

(21) Appl. No.: 19/042,303

(22) Filed: Jan. 31, 2025

Related U.S. Application Data
(60) Provisional application No. 63/627,877, filed on Feb. 1, 2024.

(51) Int. Cl. C12Q 1/6872; C12Q 1/527; G01N 30/02; G01N 30/30; G01N 30/32; G01N 30/72; G01N 30/74

(52) U.S. Cl. CPC C12Q 1/6872; C12Q 1/527; G01N 30/7233; G01N 30/74; G01N 2030/027; G01N 30/30; G01N 2030/324; G01N 2333/988

(57) ABSTRACT

The present disclosure provides methods for mapping the sequence of a polynucleotide of interest in a sample using flow-through based limited digestion. A digestive enzyme is immobilized to a solid surface, and a sample including the polynucleotide of interest is contacted to the solid surface in flow-through mode to digest the polynucleotide of interest. The flow-through fraction comprising the digested polynucleotide of interest is collected and then subjected to liquid chromatography-mass spectrometry (LC-MS) analysis to map the sequence of the polynucleotide of interest.

### CROSS REFERENCE TO RELATED APPLICATION

This application claims priority to, and the benefit of, U.S. Provisional Application No. 63/627,877, which was filed on Feb. 1, 2024, the disclosure of which is hereby incorporated by reference in its entirety.

### BACKGROUND

Messenger RNAs (mRNA) have emerged as an important class of therapeutics with great promises in treatment of various diseases including cancer, genetic and infectious diseases. As with most biotherapeutics, the sequence accuracy and integrity of mRNA is a critical quality attribute (CQA), influencing the translation efficiency and protein expression accuracy of mRNAs.

Liquid chromatography coupled with tandem mass spectrometry (LC-MS/MS), when combined with enzymatic digestion, is extremely valuable in sequence characterization of protein therapeutics. This approach, however, face limitations in achieving comprehensive sequence coverage, due to the often short and non-unique oligos produced from common nuclease digestion.

mRNA-based therapeutics must meet a high standard of quality control; thus, it is important to monitor process-related and product-related impurities that may arise during production. The development of a flow-through (FT)-based limited digestion and LC-MS/MS workflow for high-throughput sequence-coverage mapping of therapeutic mRNAs will allow for monitoring of process-related and product-related impurities.

It will be appreciated that a need exists for methods to identify and characterize mRNA therapeutics to mitigate safety risks.

### SUMMARY

The present disclosure provides methods for identifying and/or characterizing the sequence of a polynucleotide of interest in a sample, comprising:
contacting the sample comprising the polynucleotide of interest with a solid support having a digestive enzyme immobilized thereto to thereby form a digested sample;
eluting the digested sample from the solid support to form an eluate; and
analyzing the eluate by liquid chromatography-mass spectrometry, to identify and/or characterize the sequence of the polynucleotide of interest.

This disclosure provides methods for mapping the sequence of a polynucleotide of interest. In some exemplary embodiments, the methods can comprise: (a) immobilizing a digestive enzyme to a solid surface; (b) contacting a sample including the polynucleotide of interest to the solid surface in flow-through mode, wherein the digestive enzyme immobilized on the solid surface digests the polynucleotide of interest; (c) collecting a flow-through fraction comprising the digested polynucleotide of interest; and (d) subjecting the flow-through to liquid chromatography-mass spectrometry analysis to map the sequence of the polynucleotide of interest.

In one aspect, the polynucleotide of interest is a mRNA. In another aspect, the length of the mRNA is about 700 nt to about 5000 nt. In another aspect, the mRNA comprises a modified nucleoside. In yet another aspect, the nucleoside is uridine. In yet another aspect, the modified nucleoside comprises pseudouridine (ψ), N1-methylpseudouridine (m1ψ), or 5-methoxyuridine (5moU).

In one aspect, the amount of polynucleotide of interest in the sample is about 20 μg.

In one aspect, the digestive enzyme is RNase T1.

In one aspect, immobilizing, contacting, and collecting are automated.

In another aspect, immobilizing comprises contacting a biotinylated digestive enzyme to a solid surface that is coated with avidin, streptavidin, or a variant thereof. In another aspect, the solid surface is a microcartridge.

In one aspect, the amount of immobilized digestive enzyme is about 500 units to about 3000 units. In another aspect, the amount of immobilized digestive enzyme is about 1500 units.

In one aspect, the sample is contacted to the solid surface at a temperature of about 25° C. to about 45° C. In another aspect, the temperature is about 35° C.

In one aspect, the sample is contacted to the solid surface at a flow rate of about 10 μL/min to about 20 μL/min. In another aspect, the flow rate is about 10 μL/min.

In one aspect, the total time the sample is contacted to the solid surface is about 5 to 15 minutes. In another aspect, the total time is about 10 minutes.

In one aspect, the liquid chromatography is ion-pairing reversed phase liquid chromatography. In another aspect, the mass spectrometry is performed in tandem. In yet another aspect, the mass spectrometer is an electrospray ionization mass spectrometer, nano-electrospray ionization mass spectrometer, quadropole mass spectrometer, an Orbitrap-based mass spectrometer, or a hybrid quadropole-Orbitrap-based mass spectrometer,

In one aspect, the mass spectrometer is coupled to the liquid chromatography system. In another aspect, a splitter is used to coupled the mass spectrometer to the liquid chromatography system.

In one aspect, the liquid chromatography system is further coupled to an ultraviolet detector. In another aspect, the UV detector monitors absorbance at a wavelength of 260 nm.

These, and other, aspects of the present disclosure will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the present disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The summary, as well as the following detailed description, is further understood when read in conjunction with the appended drawings. For the purpose of illustrating the disclosed methods, there are shown in the drawings exemplary embodiments of the methods; however, the methods are not limited to the specific embodiments disclosed. In the drawings:

FIG. 1A shows a schematic illustration of flow-through (FT)-based limited RNase T1 digestion of mRNAs established on an AssayMAP Bravo platform. Step 1 illustrates the biotinylation of RNase T1 by NHS-Biotin. Step 2 illustrates the immobilization of biotinylated RNase T1 onto streptavidin cartridges. Step 3 illustrates the digestion of mRNA by aspirating a temperature-controlled mRNA solution through RNase T1 immobilized cartridges at a determined flow rate. Step 4 illustrates the analysis of the digested mRNA by ion-pairing reversed phase liquid chromatography-tandem mass spectrometry (IP-RP-LC-MS/MS).

FIG. 1B shows the generation of longer (and more likely unique) oligonucleotides by limited digestion compared to complete digestion.

FIG. 2 shows the effect of the amount of immobilized RNase T1 on the number of unique sequences and sequence coverage of EGFP mRNA, according to an exemplary embodiment.

FIG. 3 shows total ion chromatograms (TICs) of EGFP mRNA digests obtained by using different amounts of immobilized RNAse T1, according to an exemplary embodiment.

FIG. 4 shows the effect of the flow rate on the number of unique sequences and sequence coverage of EGFP mRNA, according to an exemplary embodiment.

FIG. 5 shows the effect of cartridge temperature (or digestion temperature) on the number of unique sequences and sequence coverage of EGFP mRNA, according to an exemplary embodiment.

FIG. 6 shows the effect of the amount of immobilized RNase T1 on the number of unique sequences and sequence coverage of Cas9-5moU mRNA, according to an exemplary embodiment.

FIG. 7 shows the effect of the flow rate on the number of unique sequences and sequence coverage of Cas9-5moU mRNA, according to an exemplary embodiment.

FIG. 8 shows the effect of cartridge temperature (or digestion temperature) on the number of unique sequences and sequence coverage of Cas9-5moU mRNA, according to an exemplary embodiment.

FIG. 9 shows the sequence coverage map of EGFP mRNA under the optimized conditions of 35° C. digestion temperature, 1500 U immobilized RNase T1, and 10 μL/min flow rate, according to an exemplary embodiment.

FIG. 10 shows a comparison of the number of identified unique oligonucleotides from FT-based limited digestion and in-solution complete digestion grouped by the number of miscleavages (0-4), according to an exemplary embodiment.

FIG. 11 shows the catalytic mechanism of RNA cleavage by RNase T1.

FIG. 12A shows a comparison of oligonucleotide products based on 3′ structural differences and the number of miscleavages from in-solution digestion, according to an exemplary embodiment.

FIG. 12B shows a comparison of oligonucleotide products based on 3′ structural differences and the number of miscleavages from FT-based limited digestion, according to an exemplary embodiment.

FIG. 13A shows an extracted ion chromatogram (EIC) of the oligoribonucleotide UUCGAGGGCG obtained by FT-based limited digestion of EGFP mRNA, according to an exemplary embodiment.

FIG. 13B shows an EIC of the oligoribonucleotide UUCGAGGGCG with the 5-methoxy uridine modification obtained by FT-based limited digestion of EGFP mRNA EGFP-5moU, according to an exemplary embodiment.

FIG. 14A shows a MS/MS spectrum of the oligoribonucleotide UUCGAGGGCG obtained by FT-based limited digestion of EGFP mRNA, according to an exemplary embodiment.

FIG. 14B shows a MS/MS spectrum of the oligoribonucleotide UUCGAGGGCG with the 5-methoxy uridine modification obtained by FT-based limited digestion of EGFP mRNA EGFP-5moU, according to an exemplary embodiment.

FIG. 15 shows a comparison of fully cleaved (grey) and miscleaved (red) products generated by FT-based limited digestion and in-solution (complete) digestion of EGFP mRNA, EGFP-moU mRNA, Cas9-5moU mRNA, and Cas9-5moU mRNA, according to an exemplary embodiment.

FIG. 16A shows TICs from triplicate analyses of RNase T1-digested EGFP mRNA using the FT-based limited digestion method, according to an exemplary embodiment.

FIG. 16B shows the repeatability of the FT-based limited digestion workflow, according to an exemplary embodiment. TICs from triplicate analyses of RNase T1-digested EGFP mRNA using the FT-based limited digestion method (replicate 1: gray; replicate 2: blue; and replicate 3: red).

FIG. 17 shows a Venn diagram depicting the overlap of identified oligonucleotides in the triplicate analyses, according to an exemplary embodiment. Each circle represents a distinct replicate, illustrating the number of unique and shared oligonucleotides identified among the replicates.

FIGS. 18A, FIG. 18B, and FIG. 18C show a comparison of RNase T1 digestion products of EGFP mRNA from FT-based limited digestion and in-solution complete digestion.

FIG. 18A shows a comparison of the number of identified oligonucleotides from both digestions grouped by the number of miscleavages (0-4) in the sequence. The number of uniquely mappable sequences within each group is also shown.

FIG. 18B shows a comparison of oligonucleotide products based on 3′ structural differences and the number of miscleavages from digestions carried out using in-solution and limited digestion workflow. “Others” represents oligonucleotides associated with the 3′-OH form by itself or together with 2′,3′-cP and 3′-P forms. The LC-MS injection amount was 10 g.

FIG. 18C shows a comparison of oligonucleotide products based on 3′ structural differences and the number of miscleavages from digestions carried out using FT-based and limited digestion workflow. “Others” represents oligonucleotides associated with the 3′-OH form by itself or together with 2′,3′-cP and 3′-P forms. The LC-MS injection amount was 10 g.

### DETAILED DESCRIPTION

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the herein disclosure. Where a range of numerical values is stated herein as being less than a stated value, the range is nevertheless bounded on its lower end by a non-zero value. It is not intended that the scope of the methods be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

Unless described otherwise, all technical and scientific terms used herein have the same meaning as commonly understood by one of ordinary skill in the art to which this disclosure belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, a “sample” refers to a mixture of molecules that comprises at least a polynucleotide of interest that is subjected to manipulation in accordance with the methods of the disclosure, including, for example, separating, analyzing, extracting, concentrating, profiling and the like.

As used herein, the terms “nucleic acid,” “polynucleotide,” or “oligonucleotide” refer a polymer composed of nucleotides or nucleosides (including ribonucleotides, deoxyribonucleotides, or analogs or modified versions thereof), which have nitrogenous heterocyclic bases or base analogs linked together along a backbone. Oligonucleotides may be isolated from genes, or chemically synthesized by methods known in the art. A nucleic acid “backbone” may be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid may be ribose, deoxyribose, or similar compounds with optional substitutions, e.g., methoxy or 2′ halide substitutions. Oligonucleotides may be DNA (deoxyribonucleic acid), RNA (ribonucleic acid), or a hybrid. Oligonucleotides may be single-stranded, double-stranded, or multi-stranded. Oligonucleotides may be of a variety of different lengths, depending on the form. Oligonucleotides often range in size from a few monomeric units, e.g. 3-4, to hundreds of monomeric units. Whenever an oligonucleotide is represented by a sequence of letters, such as “ATGCCTG,” it will be understood that the nucleotides are in 5′→3′ order from left to right and that “A” denotes adenosine, “C” denotes cytosine, “G” denotes guanosine, and “T” denotes thymidine, unless otherwise noted. The letters A, C, G, and T may be used to refer to the bases themselves, to nucleosides, or to nucleotides comprising the bases, as is standard in the art.

The term “DNA (deoxyribonucleic acid)” or “DNA molecule” refers to a chain of nucleotides comprising deoxyribonucleotides that each comprise one of four nucleobases, namely, adenine (A), thymine (T), cytosine (C), and guanine (G). The term “RNA (ribonucleic acid)” or “RNA molecule” refers to a chain of nucleotides comprising four types of ribonucleotides that each comprise one of four nucleobases, namely; A, uracil (U), G, and C. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G).

Since its discovery in the 1960s, messenger RNAs (mRNAs) have been a target of interest as therapeutic tools by researchers. Early investigation of mRNA-based therapeutics was challenging due to issues related to the stability, cell delivery efficiency, and induced immune responses of the mRNA molecules. To overcome these obstacles, significant efforts were dedicated to technological innovations, including developing modified nucleosides to reduce immunogenicity and developing lipid nanoparticles (LNPs) for cellular delivery and degradative protection of mRNA molecules. These breakthroughs were crucial for the successful development and deployment of the mRNA-based vaccines in combating the coronavirus disease 2019 (COVID-19) pandemic. Due to the success of mRNA-based vaccines, significant efforts have been dedicated to evaluating mRNA therapeutics for the treatment of various diseases, including infectious diseases, genetic diseases, cancers, and cardiovascular diseases.

mRNAs are commonly manufactured by in vitro transcription (IVT) before formulation and encapsulation with LNPs. As with other biotherapeutics, the integrity and accuracy of mRNA sequence is a critical quality attribute (CQA) due to its impact on protein translation efficiency and fidelity. Therefore, analytical characterization of the mRNA primary structure is an important task during the development and manufacturing of mRNA therapeutics. Conventionally, oligonucleotide sequencing techniques, such as Sanger sequencing and next-generation sequencing (NGS), have been used for sequence analysis of mRNAs, due to their excellent reliability, cost-effectiveness, and method throughput.

Alternatively, mass spectrometry (MS)-based approaches have been explored for the characterization of new therapeutic modalities due to their exceptional specificity, sensitivity, ability to multiplex, and quantitation performance. Recently, ion-paring reversed-phase liquid chromatography coupled with tandem MS (IP RP-LC-MS/MS) has shown great promises in sequence mapping of mRNAs. Similar to protein sequence mapping, LC-MS/MS-based mRNA sequence mapping relies on effective enzymatic digestion (commonly by endoribonucleases) of the mRNA molecules, followed by LC-MS/MS identification of the resulting oligonucleotide fragments to map the mRNA sequence.

The liquid chromatography can be ion-pairing reversed phase liquid chromatography. The mass spectrometer can be coupled to the liquid chromatography system. The liquid chromatography system can be further coupled to an ultraviolet detector. The ultraviolet detector can monitor absorbance at a wavelength of 260 nm.

The method may comprise mapping the digested polynucleotide to an undigested polynucleotide.

In this workflow, only fragments possessing unique sequences can yield useful information for protein or mRNA sequence confirmation. However, unlike proteins that are composed of 20 different amino acid residues, mRNAs only contain four nucleotide building blocks, namely, adenosine monophosphate (AMP, A), guanosine monophosphate (GMP, G), uridine monophosphate (UMP, U), and cytidine monophosphate (CMP, C). This feature renders the generation of oligonucleotide fragments with unique sequences significantly more challenging compared to that from enzymatic digestion of proteins. For example, commonly used endoribonucleases for mRNA sequence mapping, such as RNase T1 (G-specific) and MCi (U-specific), predominantly generate short oligonucleotides that are often isomeric or identical to each other. These fragments cannot be uniquely mapped to the mRNA sequence and, thereby, cannot be counted towards sequence identification.

To overcome this limitation, endonucleases with stricter cleavage specificities that produce longer (and more likely unique) oligonucleotides could be used either in parallel with RNase T1 or alone to improve the overall sequence coverage of mRNAs. For example, Tao Jiang et al., Oligonucleotide Sequence Mapping of Large Therapeutic mRNAs via Parallel Ribonuclease Digestions and LC-MS/MS. Analytical Chemistry, 2019, 91 (13), 8500-8506, reported that the combined use of colicin E5 (cleaves between GU³⁹), E. coli MazF (cleaves at the 5′ end of ACA⁴⁰), and RNase T1 results in a 1- to 2-fold increase in combined sequence coverages of three mRNAs (from 30-53% to 73-87%). Recently, Eric J. Wolf et al., Human RNase 4 improves mRNA sequence characterization by LC-MS/MS. Nucleic Acids Research 2022, 50 (18), e106-e106, showed that digestion by human RNase 4, a semi-specific enzyme that mainly cleaves between UR (R=A or G), results in improved sequence coverage of mRNA by LC-MS/MS.

Alternatively, limited digestion, which intentionally reduces RNase digestion efficiency and produces longer but unique oligonucleotides due to the occurrence of miscleavages, is another viable approach to improve the sequence mapping of mRNAs. For example, Christina J. Vanhinsbergh et al., Characterization and Sequence Mapping of Large RNA and mRNA Therapeutics Using Mass Spectrometry. Analytical Chemistry, 2022, 94 (20), 7339-7349, developed a partial digestion approach using immobilized RNase T1, which improved sequence coverages of multiple mRNAs to more than 80%. By immobilizing the RNase T1 on magnetic particles that can be easily removed from the solution, the enzymatic reaction time can be easily tuned to achieve the desired degree of digestion.

By modifying these prior methods, an improved novel flow-through (FT)-based limited RNase T1 digestion workflow was developed. In this workflow, unique oligonucleotides with miscleavages are produced to achieve ultra-high coverage for mRNA sequence mapping. The mRNA samples are then digested as they are aspirated through a solid support to which a digestive enzyme is immobilized (an exemplary embodiment of which uses an RNase T1-immobilized cartridge on the Agilent AssayMAP Bravo automation platform) under controlled temperature and flow rate. The digested samples can then be collected as the flow-through fraction and subjected to subsequent ion-pairing reversed phase liquid chromatography-tandem mass spectrometry (IP RP-LC-MS/MS) analysis. Notably, due to the flow-through nature of the digestion and its execution on an automation platform, the degree of digestion using this method can be precisely controlled, leading to excellent reproducibility. Together with its high feasibility, this method can be applied in sequence analysis for mRNA therapeutics product to ensure product quality and characterization of therapeutic mRNAs.

The present disclosure provides methods for identifying and/or characterizing the sequence of a polynucleotide of interest in a sample, comprising:
contacting the sample comprising the polynucleotide of interest with a solid support having a digestive enzyme immobilized thereto to thereby form a digested sample;
eluting the digested sample from the solid support to form an eluate; and
analyzing the eluate by liquid chromatography-mass spectrometry, to identify and/or characterize the sequence of the polynucleotide of interest.

In one exemplary embodiment, the RNA molecule is a mRNA molecule. The term “mRNA” refers to messenger RNA. The term “messenger RNA (mRNA)” refers to a type of nucleic acid, more specifically a single stranded RNA molecule that is synthesized during transcription, is complementary to one of the strands of double-stranded DNA, and serves to transmit the genetic information contained in DNA to the ribosomes for protein synthesis. The mRNA may be spliced, partially spliced or unspliced, and may be eukaryotic or prokaryotic mRNA. The mRNA may also be produced via de novo synthesis.

In an exemplary embodiment, the mRNA is produced by in vitro transcription (IVT) of a DNA molecule encoding the mRNA to be synthesized. The DNA encoding the RNA to be synthesized contains an RNA polymerase promoter to which RNA polymerase binds and initiates transcription. In one aspect, the RNA polymerase is a T7 RNA polymerase. The IVT process may produce nucleic acid byproducts, such as for example, long or short dsRNA, dsDNA or ssRNA.

Oligonucleotides may be modified, e.g., comprise a modified nucleotide, a modified internucleoside linkage, and/or a modified sugar moiety, or combinations thereof. In some embodiments, particular nucleotide modification(s) may be incorporated that render an oligonucleotide more resistant to nuclease digestion than the native oligoribonucleotide or oligodeoxynucleotide molecules; such modified polynucleotides survive intact for a longer time than unmodified polynucleotides. Exemplary modified polynucleotides include those comprising modified uridine, for example, pseudouridine (ψ), N1-methylpseudouridine (m1ψ), or 5-methoxyuridine (5moU). Exemplary modified nucleotides also include those comprising modified backbones, for example, modified internucleoside linkages such as, methyl phosphonates, phosphotriesters, phosphorothioates short chain alkyl or cycloalkyl intersugar linkages heterocyclic intersugar linkages or short chain heteroatomic. As such, the oligonucleotide may be stabilized against nucleolytic degradation, e.g., via incorporation of a modification, e.g., a nucleotide modification.

The amount of polynucleotide of interest in the sample can be about 20 μg. The amount of polynucleotide of interest in the sample can be about 5 μg, about 10 μg, about 20 μg, about 30 μg, or about 40 μg.

In some exemplary embodiments, the sample can be prepared prior to LC-MS analysis. Preparation steps can include reduction, denaturation, alkylation, dilution, digestion, and separation (for example, centrifugation).

As used herein, the terms “denaturing” or “denaturation” refers to a process in which the secondary structure of an oligonucleotide is disrupted, such that the double-stranded oligonucleotide is transformed into two complimentary single-stranded oligonucleotides. Denaturation may be full or partial. Denaturation may be performed by a variety of methods including heating double-stranded nucleic acid molecules (e.g., by heating the sample to 85° C. or higher), treating double-stranded nucleic acid molecules with one or more organic solvents (e.g., 0.5M NaOH, DMSO, formamide, urea, etc.), changing the salt concentration of double-stranded nucleic nucleus molecules, and/or changing the pH of double-stranded nucleic acid molecules. Denaturation may also be performed through enzymatic denaturation, such as through the use of helicases, or other enzymes with helicase activity. Oligonucleotides may also be denatured through interaction with a surface or by a physical process such as stretching beyond a critical length. As used herein, the terms “digesting” or “digestion” refers to hydrolysis of one or more phosphodiester bonds in the backbone of an oligonucleotide. Digestion may be performed by physical methods, such as sonication or physical shear, by chemical methods, such as an alkaline compound, piperidine formate, piperidine, dimethyl sulfate, hydrazine, sodium chloride, or combinations thereof, or by enzymatic digestion with a nuclease. The nuclease may be an endonuclease that cleaves the phosphodiester bond within the polynucleotide chain or an exonuclease that cleaves the phosphodiester bond at the end of the polynucleotide chain. Nucleases may cleave non-specifically or at specific sites of the oligonucleotides. Nonlimiting examples of nucleases include non-specific deoxyribonuclease I (DNaseI), ribonuclease A or T1 (RNAse A or T1), and restriction endonucleases. The digestive enzyme can be RNase T1.

Conventional methods use a digestive enzyme in conditions and concentrations sufficient to completely digest oligonucleotides in a sample prior to LC-MS analysis. The present disclosure finds that identification and characterization of mRNA fragments can be improved through limited digestion, meaning that digestion conditions are selected such that the oligonucleotide is not completely digested. In some exemplary embodiments, mRNA samples are subjected to digestion by RNase T1 without prior denaturation. In some exemplary embodiments, an amount of RNase T1 is selected to ensure limited digestion. In some exemplary embodiments, the amount of RNase T1 is about 500 units (U) to about 3000 U, or about 600 U, about 700 U, about 800 U, about 900 U, about 1000 U, about 1100 U, about 1200 U, about 1300 U, about 1400 U, about 1500 U, about 1600 U, about 1700 U, about 1800 U, about 1900 U, about 2000 U, about 2100 U, about 2200 U, about 2300 U, about 2400 U, about 2500 U, about 2600 U, about 2700 U, about 2800 U, about 2900 U, or about 3000 U. The amount of immobilized digestive enzyme (e.g., RNAse T1) can be about 500 units to about 3000 units. In some exemplary embodiments, the amount of RNase T1 is 1500 U.

The polynucleotide of interest can be a DNA, RNA, mRNA, tRNA, rRNA, or hybrid thereof.

The length of the mRNA can be about 700 nucleotides to about 5000 nucleotides.

The mRNA may comprise a modified nucleoside. The mRNA may comprise a modified uridine. The modified nucleoside can be a pseudouridine (ψ), N1-methylpseudouridine (m1ψ), or 5-methoxyuridine (5moU).

In some exemplary embodiments, the digestive enzyme is immobilized on a solid support, for example, through a biotin-streptavidin interaction, and enzymatic digestion is performed by contacting the oligonucleotide to the solid support in flow-through mode, and collecting a flow-through fraction (i.e., eluate) comprising the digesting oligonucleotide. In some embodiments, the immobilizing, contacting, and collecting steps may be performed using an automated liquid handling platform, such as the AssayMAP Bravo platform.

The effective enzyme-substrate contact time (digestion time) may be affected by the sample flow rate. In some embodiments, the sample flow rate may be about 7 μL/min to about 20 μL/min, or about 7 μL/min, about 9 μL/min, about 10 μL/min, about 11 μL/min, about 12 L/min, about 13 μL/min, about 14 μL/min, about 15 μL/min, about 16 μL/min, about 17 L/min, about 18 μL/min, about 19 μL/min, or about 20 μL/min.

The sample can be contacted to the solid support at a flow rate of about 7 μL/min to about 20 μL/min. The effective enzyme-substrate contact time (digestion time) may be affected by the sample flow rate. In some exemplary embodiments, the sample flow rate may be about 7 L/min to about 20 μL/min. In some exemplary embodiments, the sample flow rate is 7 μL/min. In some exemplary embodiments, the sample flow rate is 10 μL/min. In some exemplary embodiments, the sample flow rate is 20 μL/min. Without wishing to be bound by theory, a lower sample flow rate (longer reaction time) could lead to overdigestion of the mRNA, generating predominantly short and non-unique oligonucleotides. Conversely, a sample flow rate that is too high could lead to insufficient digestion, producing oligonucleotides that are too long to be effectively identified by MS/MS.

In some exemplary embodiments, the methods comprise identifying and/or characterizing the sequence of a polynucleotide of interest in a sample, comprising: contacting the sample comprising the polynucleotide of interest with a solid support having a digestive enzyme immobilized thereto to thereby form a digested sample; eluting the digested sample from the solid support to form an eluate; and analyzing the eluate by liquid chromatography-mass spectrometry, to identify and/or characterize the sequence of the polynucleotide of interest, wherein the flow rate is about 10 μL/min.

The total reaction time the sample is contacted to the solid support can be about 5 to 15 minutes. In some exemplary embodiments, the total reaction time may be about 5 min, about 7 min, about 9 min, about 10 min, about 11 min, about 13 min, or about 15 min. In some exemplary embodiments, the total reaction time is 10 min.

In some exemplary embodiments, the methods comprise identifying and/or characterizing the sequence of a polynucleotide of interest in a sample, comprising: contacting the sample comprising the polynucleotide of interest with a solid support having a digestive enzyme immobilized thereto to thereby form a digested sample; eluting the digested sample from the solid support to form an eluate; and analyzing the eluate by liquid chromatography-mass spectrometry, to identify and/or characterize the sequence of the polynucleotide of interest, wherein the total reaction time is about 10 min.

The contact time between the immobilized enzyme and the polynucleotide may be from about 15 to about 60 seconds. In some exemplary embodiments, the contact time is about 15 seconds, about 20 seconds, about 25 seconds, about 30 seconds, about 40 seconds, about 50 seconds, or about 60 seconds. In some exemplary embodiments, the contact time is 30 seconds.

In some exemplary embodiments, the methods comprise identifying and/or characterizing the sequence of a polynucleotide of interest in a sample, comprising: contacting the sample comprising the polynucleotide of interest with a solid support having a digestive enzyme immobilized thereto to thereby form a digested sample; eluting the digested sample from the solid support to form an eluate; and analyzing the eluate by liquid chromatography-mass spectrometry, to identify and/or characterize the sequence of the polynucleotide of interest, wherein the polynucleotide is contacted with the digestive enzyme for about 30 seconds.

In some exemplary embodiments, enzymatic digestion is performed in a buffer solution, most preferably in Tris-HCl. In some exemplary embodiments, the concentration of Tris-HCl is about 10 mM, about 20 mM, about 30 mM, about 40 mM, about 50 mM, about 60 mM, about 70 mM, about 80 mM, about 90 mM, or about 1000 mM. In some exemplary embodiments, the pH of the buffer solution is about pH 7.0, about pH 7.5, about pH 8.0, or about pH 8.5. Digestion may also be carried out in ambient (room) temperature or above ambient temperature. In some exemplary embodiments, digestion is carried out at about 25° C. to about 45° C., or about 25° C., about 27° C., about 30° C., about 33° C., about 35° C., about 37° C., about 40° C., about 43° C., or about 45° C.

The sample can be contacted to the solid support at a temperature of about 25° C. to about 45° C. The sample may comprise the polynucleotide of interest and the solid support may have a digestive enzyme (e.g., RNAse T1) immobilized thereto. Contact between the sample and immobilized RNAse T1 may digest the polynucleotide in the sample. Digestion may be carried out in ambient (room) temperature or above ambient temperature. In some exemplary embodiments, digestion is carried out at about 25° C. to about 45° C. In some exemplary embodiments, digestion is carried out at 35° C.

In some exemplary embodiments, the methods comprise identifying and/or characterizing the sequence of a polynucleotide of interest in a sample, comprising: contacting the sample comprising the polynucleotide of interest with a solid support having a digestive enzyme immobilized thereto to thereby form a digested sample; eluting the digested sample from the solid support to form an eluate; and analyzing the eluate by liquid chromatography-mass spectrometry, to identify and/or characterize the sequence of the polynucleotide of interest, wherein the digestion is carried out at about 35° C.

As used herein, the term “liquid chromatography” refers to a process in which a biological and/or chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography. In some aspects, the sample or eluate can be subjected to any one of the aforementioned chromatographic methods or a combination thereof.

In some exemplary embodiments, the liquid chromatography can be ion-pairing reversed phase liquid chromatography. The term “ion-pairing reversed-phase liquid chromatography” refers to a specific form of reversed-phase liquid chromatography in which an ion with a lipophilic residue and positive charge such as an alkylammonium salt, e.g. triethylammonium acetate, is added to the mobile phase as counter ion for the negatively charged oligonucleotide. When used with common hydrophobic mobile phases in the reversed-phase mode, ion pair reagents can be used to selectively increase the retention of the oligonucleotide.

As used herein, the term “mass spectrometry” includes the use of a device capable of identifying specific molecular species and measuring their accurate masses. The term is meant to include any molecular detector into which an oligonucleotide may be characterized. The mass spectrometer can be coupled to a liquid chromatography system, wherein the mass spectrometer is capable of performing LC-MS (liquid chromatography-mass spectrometry). A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application. Non-limiting examples of ion sources include electrospray ionization (ESI), atmospheric pressure ionization (API), matrix assisted laser desorption ionization (MALDI), laser desorption ionization (LDI), and desorption electrospray ionization (DESI). The term “mass analyzer” refers to a device that can separate species, that is, atoms, molecules, or clusters, according to their mass. Non-limiting examples of mass analyzers include time-of-flight (TOF), magnetic/electric sector, quadrupole mass filter (Q), quadrupole ion trap (QIT), orbitrap, Fourier transform ion cyclotron resonance (FTICR), and also the technique of accelerator mass spectrometry (AMS).

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. MS/MS, or MS2, can be performed by first selecting and isolating a precursor ion (MS1), and fragmenting it to obtain meaningful information. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The solid support can be beads or a resin that are in a column or device that allows the sample to “flow through” the sold support. The solid support can be a microcartridge.

The method may comprise applying the sample to the solid support such that the sample flows through the solid support and forms the digested sample.

Digestion of mRNAs, when executed under limited digestion conditions, can be used to produce more miscleavage-containing oligonucleotides that are more likely to be uniquely mappable to the mRNA sequence, thereby improving the overall coverage of mRNA sequence mapping. However, as RNase T1 cannot be readily inactivated by either high temperature or common denaturants, its effective removal at the desired digestion end point is a challenge in developing a reliable limited digestion method.

To enable precise control of enzyme-substrate contact time, the disclosed methods provide a limited digestion using a Flow Through (FT)-based approach by passing the mRNA samples through a solid matrix immobilized with a digestive enzyme.

Without wishing to be bound by theory, due to the flow through nature of the digestion and its execution on an automation platform, the degree of digestion using the methods disclosed herein can be precisely controlled, leading to robust consistency.

In some embodiments, the volume of the solid support cartridge is fixed (5 L), and thus the contact time between the enzyme and the polynucleotide substrate can be controlled by modulating the flow rate of the mRNA sample solution through the solid support cartridge.

The FT-based digestion of the present disclosure can generate high abundances of miscleavage-containing oligonucleotides with unique sequence information, achieving a high sequence coverage (>96% for EGFP mRNA). In contrast to the conventional in-solution digestion method, the FT-based limited digestion method of the present disclosure can produce primarily homogeneous 2′,3′-cyclic phosphorylated oligonucleotide products, which are suitable for MS detection. The FT-based limited digestion methods of the present disclosure can be universally applied to chemically modified mRNAs and mRNAs of different lengths (up to 4245 nt tested), consistently achieving high sequence coverage (>93%).

The methods can further comprise, prior to contacting, immobilizing a digestive enzyme on a solid support. Therefore, discussed herein are methods for identifying and/or characterizing the sequence of a polynucleotide of interest in a sample, comprising: immobilizing a digestive enzyme to a solid support; contacting the sample comprising the polynucleotide of interest with the solid support to thereby form a digested sample; eluting the digested sample from the solid support to form an eluate; and analyzing the eluate by liquid chromatography-mass spectrometry, to identify and/or characterize the sequence of the polynucleotide of interest.

It is understood that the present disclosure is not limited to any of the aforesaid nucleic acid(s), digestive enzyme(s), digestion condition(s), liquid chromatography method(s), mass spectrometer(s), pH range(s) or value(s), temperature(s), or concentration(s), and any nucleic acid(s), digestive enzyme(s), digestion condition(s), liquid chromatography method(s), mass spectrometer(s), pH range(s) or value(s), temperature(s), or concentration(s) can be selected by any suitable means.

The present disclosure will be more fully understood by reference to the following examples. They should not, however, be construed as limiting the scope of the disclosure.

### EXAMPLES

Methods and Materials. Deionized water was provided by a Milli-Q integral water purification system installed with a MilliPak Express 20 filter (MilliporeSigma, Burlington, MA). Acetonitrile (Optima™ LC/MS Grade), RNase T1 (1000 U/μL), EZ-Link™ NHS-Biotin, Dimethyl sulfoxide (DMSO, Anhydrous), EDTA (0.5 M, pH 8.0, RNase-free), UltraPure™ 1 M Tris-HCl Buffer (pH 7.5), Gibco™ 1× Phosphate Buffered Saline (PBS, pH 7.4), and Pall Lab Nanosep Centrifugal Devices with Omega™ Membrane-3K was purchased from Thermo Fisher Scientific (Waltham, MA). 1,1,1,3,3,3-hexafluoro-2-propanol (HFIP, LC-MS LiChropur™) and N, N-diisopropylethylamine (DIPEA, purified by redistillation, 99.5%) was purchased form Sigma-Aldrich (St. Louis, MO). All oligonucleotide samples including EGFP mRNA, EGFP-5moU mRNA, EPO-5moU mRNA, Cas9-5moU mRNA were purchased from Trilink Biotechnologies (San Diego, CA). AssayMAP Streptavidin (SA-W) cartridge (5 μL) was obtained from Agilent Technologies (Santa Clara, CA).

Biotinylation of RNase T1. The biotinylated RNase T1 was prepared by mixing 25 μL of RNase T1 (1000 U/μL), 25 μL of NHS-biotin solution (10 mM in DMSO) and 400 μL of PBS (1×, pH 7.4), followed by incubation for 30 mins at 25° C. with shaking at 650 rpm. The reaction was quenched by adding 50 μL of 1M Tris-HCl (pH 7.5). The quenched reaction mixture was centrifuged at 14000×g in a Nanosep centrifugal device (3K) for 25 min to remove the excess NHS-biotin reagent. The recovered biotinylated RNase T1 was resuspended by adding 400 μL of PBS (1×, pH 7.4) and the final concentration was measured using a NanoDrop spectrophotometer at absorbance of 280 nm.

Immobilization of biotinylated RNase T1 on AssayMAP Cartridges. Immobilization of biotinylated RNase T1 on the streptavidin cartridges was performed on the AssayMap Bravo platform using the “immobilization” application. The priming/equilibration buffer, cartridge wash buffer and syringe wash buffer were prepared by mixing 4 mL 0.5M EDTA, 100 mL 1M Tris-HCl (pH 7.5) and 896 mL Milli Q Water together. The biotinylated RNase T1 solution (1-30 U/μL) was prepared in 1×PBS (pH 7.4). Immobilization of biotinylated RNAse T1 was performed sequentially with the following steps: 1) streptavidin cartridges were primed and equilibrated with 100 μL priming/equilibration buffer respectively; 2) 100 μL biotinylated RNase T1 solution of varying concentrations were loaded at a flow rate of 5 μL/min; 3) the cartridges were washed with 100 μL of cartridge wash buffer to remove excess/non-binding biotinylated RNase T1. The tested loading amount of biotinylated RNase T1 on the AssayMAP cartridges ranged from 100-3000 U.

Flow through (FT)-based limited digestion of mRNAs. FT-based limited digestion of mRNA was executed using the “on-cartridge reaction” application on AssayMAP Bravo platform, which enables the automated aspiration of a temperature-controlled mRNA solution through immobilized AssayMAP cartridges for limited digestion. The equilibration/chase buffer, cartridge wash buffer and syringe wash buffer were prepared by mixing 4 mL 0.5M EDTA, 100 mL 1M Tris-HCl (pH 7.5), and 896 mL Milli Q Water. The mRNA solution (0.2 μg/μL) was then prepared in the equilibration/chase buffer. On-cartridge digestion was performed sequentially with the following steps: 1) RNase T1 immobilized cartridges were re-equilibrated with 100 μL equilibration buffer, 2) 100 μL mRNA solution was aspirated onto the cartridge at a fixed temperature (10, 25, 35, and 45° C.) and flow rate (6.7, 10 and 20 μL/min). The digestion temperature (25, 35, and 45° C.) was controlled through a 96 red PCR plate thermal insert on the Peltier thermal station where the mRNA samples were seated on, so that the actual digestion temperature was slightly lower than the preset station temperature. The digestion temperature of 10° C. was controlled by storing the mRNA samples and the PCR thermal insert plate in the 4° C. refrigerator prior to limited digestion, which returned to approximately 10° C. during the digestion process under room temperature. The aspiration flow rate was controlled by the total reaction time (5-15 mins), and 3) The reaction was stopped by loading 5 μL of chase buffer. The combined flow-through fraction containing mRNA digestion products was collected for LC-MS/MS analysis.

In-solution complete digestion of mRNAs. In-solution digestion of mRNAs was performed by mixing 20 μL mRNA or mRNA mixtures (1 μg/μL) with 30 μL of 8 M Urea, 6 μL of 1 M Tris-HCl (pH 7.5) and 0.4 μL 0.5 M EDTA, and incubating the mixture at 80° C. for 10 min for sample denaturation. The denatured sample was then cooled to room temperature before it was mixed with 10 μL of 1000 U/μL RNase T1, followed by incubation at 37° C. for 15 min. After digestion, 34 μL PBS (1×, pH 7.4) solution was added to dilute the mRNA solution to 0.2 μg/μL prior to LC-MS/MS analysis.

Ion-pairing reversed phase-liquid chromatography-tandem mass spectrometry (IP-RP-LC-MS/MS) analysis of mRNA digests. IP RP-LC separation of mRNA digests was performed on a Waters ACQUITY H-Class UPLC equipped with an ACQUITY UPLC oligonucleotide BEH C18 (130 Å, 1.7 m, 2.1 mm×150 mm) column with a column temperature of 60° C. Mobile phase A (MPA) consisted of 1% HFIP and 0.1% DIPEA in H₂O, and mobile phase B (MPB) consisted of 0.075% HFIP and 0.0375% DIPEA in 65% ACN and 35% H₂O. The LC gradient operated at a flow rate of 0.35 mL/min was set as follows: 0 min, 3% B; 5 min, 3% B; 20 min, 15% B; 23 min, 25% B; 23.01 min, 3% B; 25 min, 3% B; 27 min, 95% B; 34 min, 95% B; 34.01 min, 3% B; 37 min, 3% B.

The post-LC flow was subjected to online UV detection at a wavelength of 260 nm followed by MS/MS analysis performed on a Thermo Q-Exactive plus mass spectrometer equipped with a heated electrospray ion source (HESI). The instrument HESI source parameters were set as follows: spray voltage 3.0 kV negative polarity, sheath gas 40 (arbitrary units), Aux gas 10, capillary temperature 275° C., Aux gas temperature 350° C., and S-lens RF level 65. MS analysis was performed in the data-dependent acquisition mode. The full MS scan was performed with an instrument resolving power set to 35,000, scan range 500-3000, maximum IT 50 ms, and AGC target 3E6. For data dependent MS2 analysis, the following scan parameters were used: NCE 24, resolving power 17,500, scan range 200-2000, maximum IT 100 ms, AGC target 5E4, dynamic exclusion 5 s.

Data analysis. Data processing was performed using Byonic software from Protein Metrics (Cupertino, CA). The digestion and instrument parameters were set as follows: The cleavage site—G, cleavage side—C-terminal, digestion specificity—fully specific (limiting cleavage to only the 3′ side of guanosine residues in the RNA), maximum missed cleavages—4, precursor mass tolerance—20 ppm, fragment mass tolerance—50 ppm, fragmentation type-NUVPD. For the fixed and variable modification settings, hydroxylation of the 5′ terminal (−79.9663 Da) was set as fixed, and phosphorylation (+79.9663 Da) and cyclic phosphorylation (+61.9558 Da) of the 3′ terminal was set as variable common modifications. For analyzing mRNAs fully substituted with 5-methoxyuridine, 5-methoxyuridine (+30.0106 Da) was added as a fixed modification. The maximum number of common modifications was set to 1. The maximum precursor mass was set as 10,000 Da, and the oligonucleotide output option was set with a score cutoff threshold of 275.

### Example 1. Optimization of Flow-Through (FT)-Based Limited RNaseT1 Digestion

RNase T1 digestion of mRNAs, when executed under limited digestion conditions, can be used to produce miscleaved oligonucleotides that are more likely to contain unique sequences and thus improve the coverage of mRNA sequence mapping. However, as RNase T1 cannot be readily inactivated by either high temperature or common denaturants, its effective removal at desired digestion end point is key to the success and has been the biggest challenge in developing a reliable limited digestion method.

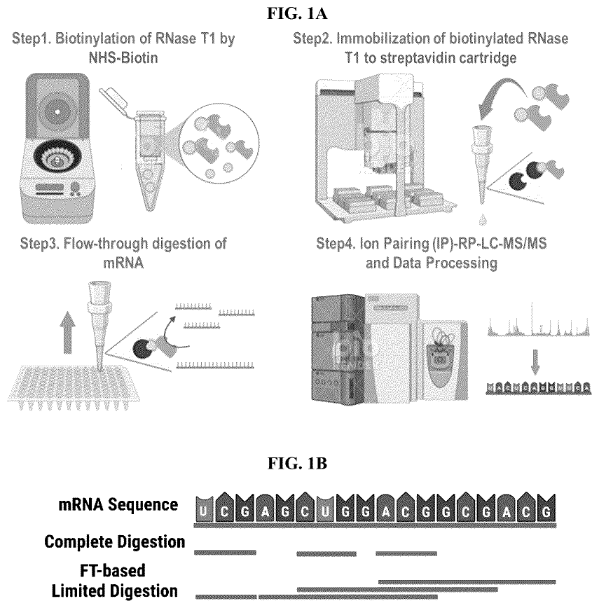

To enable precise control of enzyme-substrate contact time, limited digestion was executed using a FT-based approach by passing the mRNA samples through a solid matrix immobilized with RNase T1. This was achieved on an AssayMAP Bravo automation platform to take advantage of the high precision and multi-channel capabilities of this liquid handling system. In this workflow, RNase T1 was first biotinylated and immobilized onto the streptavidin cartridges using streptavidin-biotin chemistry (FIG. 1A). Subsequently, the mRNA sample prepared in digestion buffer was aspirated through the cartridge at a controlled flow rate and temperature (FIG. 1A). Finally, the digested mRNA is analyzed IP-RP-LC-MS/MS. Notably, as the volume of each cartridge is fixed (5 μL), the contact time between the enzyme and the substrate can be precisely controlled by modulating the mRNA sample solution flow rate through the cartridge. By optimizing the enzyme-substrate contact time, the digestion temperature, and the amount of immobilized enzyme, the RNase T1 digestion efficiency can be fine-tuned to produce increased levels of miscleaved oligonucleotides, thereby achieving higher sequence coverage of mRNAs (FIG. 1B).

Three major factors that may impact the digestion efficiency the amount of immobilized RNase T1 (or enzyme-to-substrate ratio when fixed amount of mRNA is used), the reaction time (or flow rate), and the reaction temperature-were evaluated using EGFP mRNA (720 nt) as a model system. First, to optimize the amount of immobilized RNase T1 for each digestion, four sets of immobilized cartridges were prepared by loading 100 U, 500 U, 1500 U, and 3000 U of RNase T1, respectively, onto each cartridge during the immobilization step. Each cartridge was used to digest 100 μL of EGFP mRNA (0.1 μg/μL) by aspirating the sample solutions through the cartridges at a flow rate of 10 μL/min. Throughout the digestion process, the mRNA samples were seated on a Peltier thermal station with the station temperature kept at 45° C. (the actual digestion temperature being slightly lower). The digestion products were then analyzed by LC-MS/MS to identify the generated oligonucleotides.

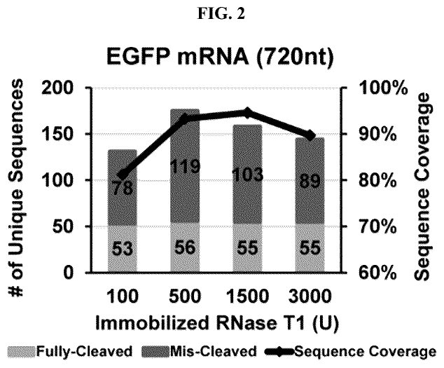

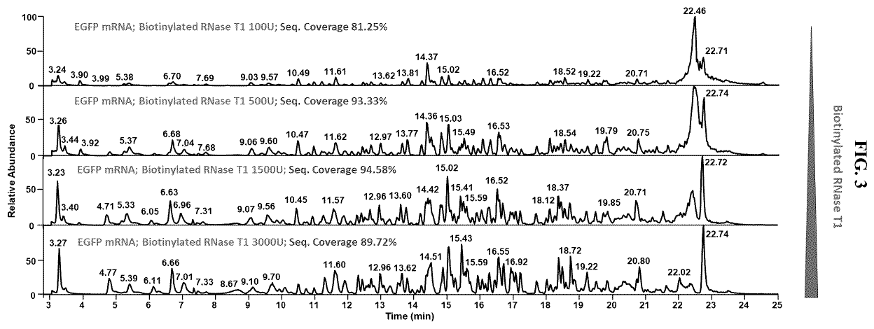

Since the goal of applying limited digestion is to improve mRNA sequence coverage by producing more miscleaved (or unique) oligonucleotides, the number of identified oligonucleotides with unique sequences, as well as the overall sequence coverage, were used as key indicators for method evaluation (FIGS. 2, 4, and 5). Further, the total number of unique oligonucleotides were categorized into two groups, including the fully cleaved (grey) and mis-cleaved products (red), respectively, to better understand the impact of experimental conditions on digestion efficiency. As shown in FIG. 2, digestion by 500-1500 U biotinylated RNase T1 resulted in the most unique oligonucleotide sequences and the highest sequence coverage of EGFP mRNA. In contrast, when the amount of RNase T1 was decreased to 100 U or increased to 3000 U, both the number of unique oligonucleotides and sequence coverage dropped. This is presumably attributed to insufficient or over digestion at low or high enzyme-to-substrate ratios, respectively.

Additionally, comparisons of the total ion chromatograms (TICs) from IP RP-LC-MS/MS analyses of the four different digests also revealed a scarcity of resulting oligonucleotides in the 100 U digestion sample, agreeing with the expected low digestion efficiency under this condition (FIG. 3). Meanwhile, an increasing trend in the relative abundances of early-eluting oligonucleotides was observed in samples digested with an increasing amount of RNase T1. It is worth noting that IP RP-LC separates oligonucleotide fragments primarily in a size-based order, with shorter oligonucleotides eluting earlier than longer oligonucleotides that often contain more miscleavages. Therefore, the increasing abundance of short oligonucleotides in the presence of a higher enzyme amount was consistent with the expectation that more complete digestion should occur under such conditions.

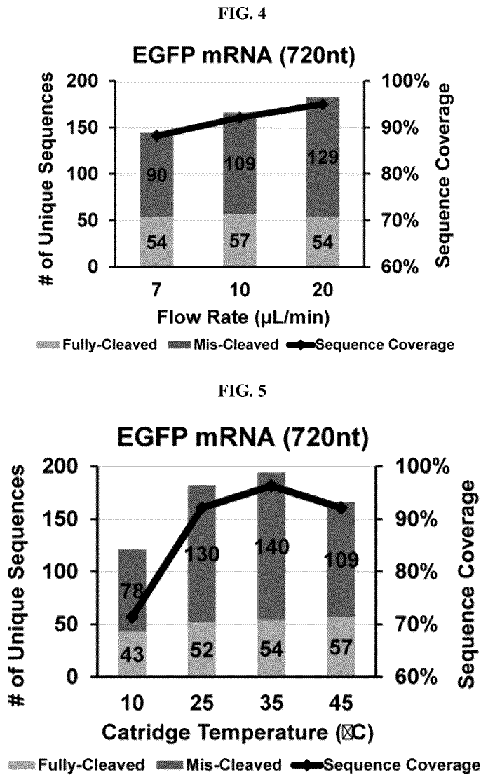

Secondly, the on-cartridge reaction time was also optimized. Considering the nature of the FT-based digestion, the effective enzyme-substrate contact time (digestion time) was directly determined by the sample flow rate. Therefore, three different sample flow rates, including 7, 10, and 20 μL/min, were evaluated for the digestion of EGFP mRNA, while the amount of RNase T1 and digestion station temperature were fixed at 1500 U and 45° C., respectively. As shown in FIG. 4, a concomitant increase in both sequence coverage and number of unique oligonucleotides was observed with the increase of flow rate from 7 to 20 μL/min. This is consistent with the expectation that a lower sample flow rate (longer reaction time) could lead to over-digestion of the mRNA, generating predominantly short and non-unique oligonucleotides. On the other hand, a sample flow rate that is too high could lead to insufficient digestion, producing oligonucleotides that are too long to be effectively identified by MS/MS. Under the tested ranges, it was found that both flow rates at 10 or 20 μL/min could produce desired levels of mis-cleaved oligonucleotides and achieve high sequence coverage (>90%) of EGFP mRNA. Notably, considering the bed volume of each cartridge was 5 p L, the equivalent enzyme-substrate contact time at the sample flow rate of 10 μL/min was only 30 seconds. This extremely short reaction time highlights the necessity of precise method execution using the automation platform, as the alternative manual approach is expected to face significant challenges in achieving the required accuracy and consistency.

Similarly, the impact of digestion temperature was investigated by evaluating the digestion outcome at four sample station temperatures (10° C., 25° C., 35° C., and 45° C.), using the previously optimized RNase T1 amount (1500 U) and sample flow rate (10 μL/min). As shown in FIG. 5, a digestion temperature of 10° C. resulted in the least digestion products and the poorest sequence coverage compared to the other three conditions, presumably due to decreased catalytic activity of RNase T1 at low temperature. On the other hand, digestion carried out at 35° C. compared slightly more favorably to 25° C. and 45° C., which resulted in the most unique oligonucleotide sequences and the highest achieved sequence coverage of EGFP mRNA.

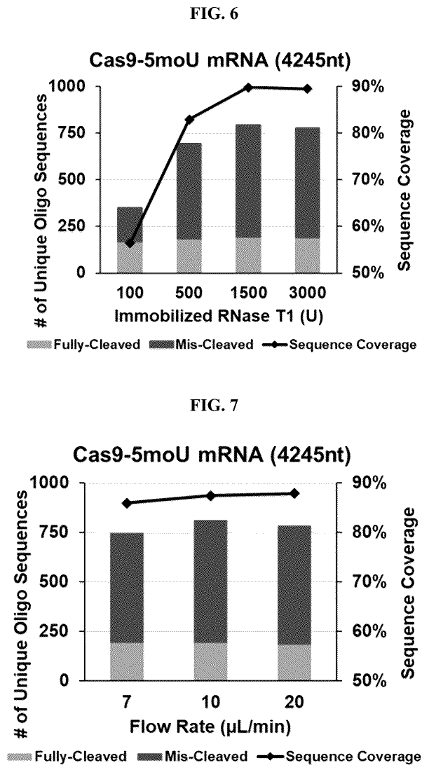

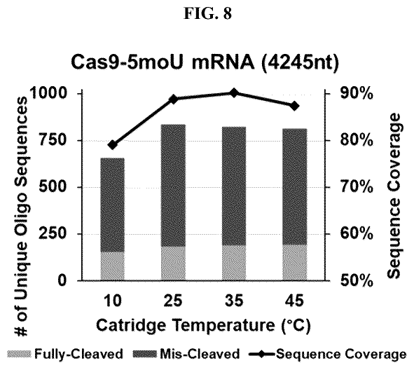

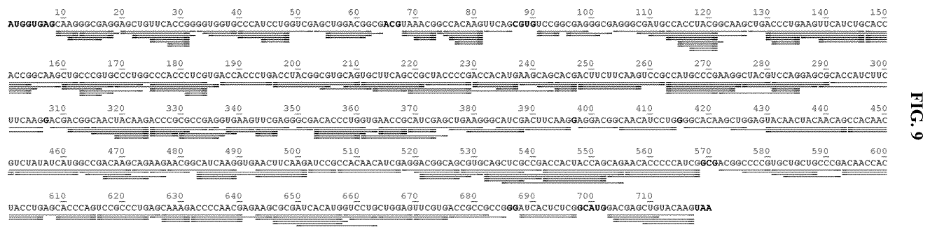

In addition to EGFP mRNA, another mRNA of approximately 6× length (Cas9, >4000 nucleotides) was also utilized in similar method optimization experiments to evaluate the impact of mRNA size on optimal digestion parameters (FIGS. 6-8). Interestingly, despite the significant differences in their lengths, the optimal digestion conditions identified for both EGFP mRNA and Cas9 mRNA were nearly identical. In particular, both mRNAs required an optimal digestion temperature of 35° C. and 1500 U of immobilized RNase T1 to achieve the highest sequence coverage (FIG. 6). However, while digestion of EGFP mRNA at a sample flow rate of 20 μL/min produced slightly better sequence coverage than 10 μL/min (FIG. 7), both conditions yielded similar performance for the digestion of Cas9 mRNA (FIG. 8).

To ensure general applicability and robust execution, a slightly lower sample flow rate of 10 μL/min, along with a digestion temperature of 35° C. and 1500 U of immobilized RNase T1, were selected as the platform conditions for FT-based limited digestion of mRNAs. Under these conditions, FT-based limited digestion of EGFP mRNA resulted in a total of 194 unique oligonucleotides, including 54 fully cleaved products and 140 miss-cleaved products, and achieving an overall sequence coverage of 96.3% (FIG. 9). This achieved sequence coverage represents an unprecedented improvement from those previously reported using conventional complete digestion or limited digestion protocols, highlighting the great potential of this method in mRNA sequence mapping.

### Example 2. Characterization of EGFP mRNA by FT-Based Limited RNaseT1 Digestion

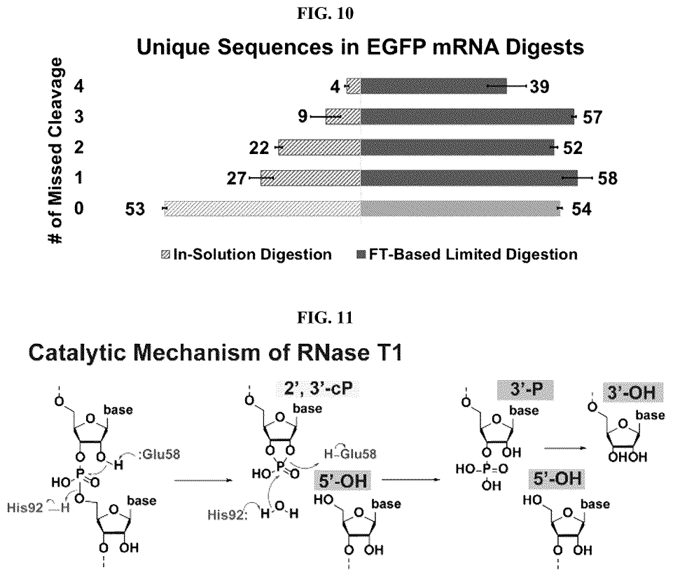

To gain a comprehensive understanding of the FT-based RNase T1 digestion and its improvement on sequence coverage, the digestion products of EGFP mRNA were thoroughly characterized and compared with those from conventional solution-based digestion. The LC-MS/MS-identified oligonucleotides were first screened for unique sequences, and then grouped by the number of miscleavages in the sequence (FIG. 10). Triplicate analyses revealed that a total of 261±4 unique oligonucleotides were identified from FT-based limited digestion, of which 54±1 were fully digested (0 miscleavage) and 207±3 contained 1-4 miscleavages. Notably, longer oligonucleotides with more than 4 miscleavages were also observed in the digested samples. However, these oligonucleotides were outside the detectable range by MS/MS, and therefore, not included. Together, these identified oligonucleotides accounted for an overall sequence coverage of 96.5%±1.1% for EGFP mRNA by FT-based limited digestion. In comparison, solution-based digestion produced a similar number of fully digested oligonucleotides (53±1) but significantly fewer miscleaved products (62±4), leading to much lower sequence coverage (79.8%±2.2%). These results highlight the critical role of miscleaved oligonucleotides in improving mRNA sequence mapping and supporting the use of limited digestion strategies for mRNA sequencing.

Like other endoribonucleases, RNase T1 digestion of mRNAs often produces a mixture of digestion products, which may include 2′, 3′-cyclic phosphorylated, 3′-phosphorylated, and 2′, 3′-hydroxylated forms (FIG. 11). 2′, 3′-cyclic phosphate (2′, 3′-cP) is an intermediate species generated by the initial RNase T1 digestion, known as transphosphorylation. Subsequently, 2′, 3′-cP intermediates could undergo hydrolysis and form 3′-phosphate (3′-P) products. As transphosphorylation occurs much faster than the hydrolysis reaction, it is common to find both 2′, 3′-cP and 3′-P products in the final digested sample. Although less common, 3′-hydroxylated (3′-OH) oligonucleotides could also be produced via further hydrolysis of the 3′-P species. This heterogeneity of digestion products, arising from different phosphorylation structures, could potentially convolute and reduce the sensitivity of MS analysis. Therefore, the different phosphorylation structures were also evaluated for both FT-based limited digestion and solution-based digestion products.

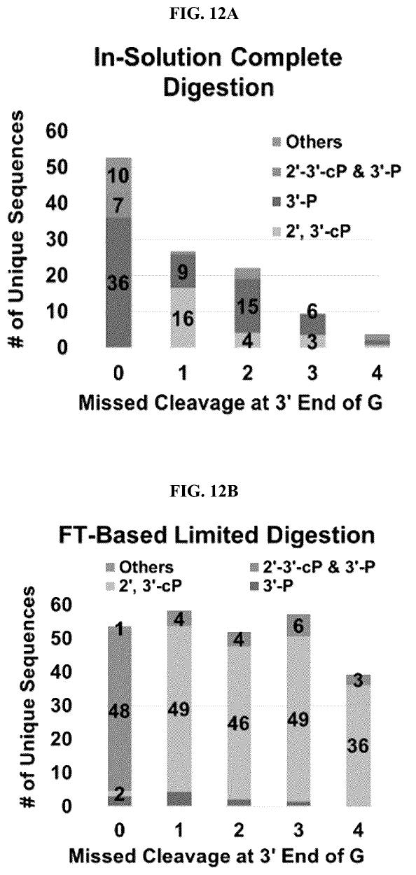

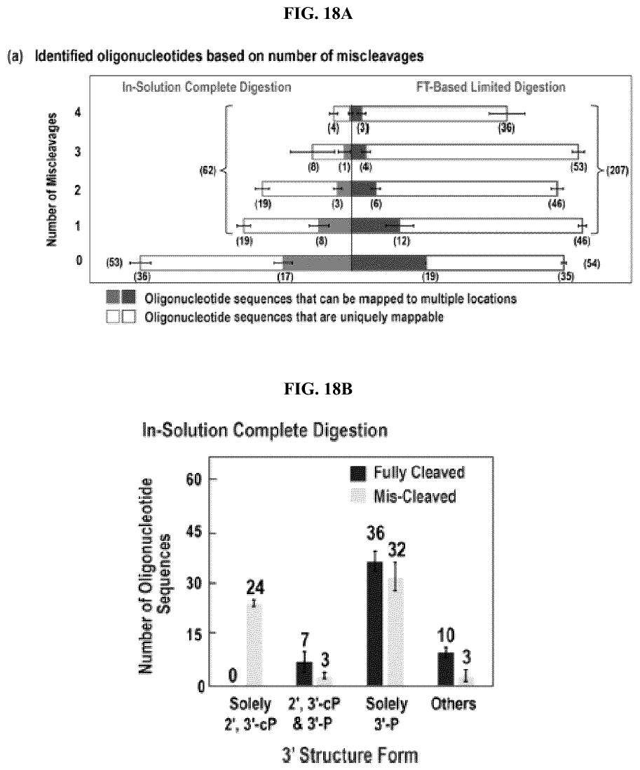

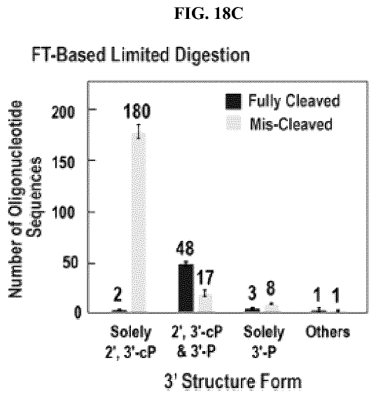

Analysis of the solution-based digestion products of EGFP mRNA (FIG. 12A and FIG. 18B) showed that the majority of the resulting unique oligonucleotides adopted a single 3′-P form ( 68/114), while smaller populations existed solely in the 2′, 3′-cP form ( 24/114) or in both 2′,3′-cP and 3′-P forms (10/114). The remaining 12 unique oligonucleotides were found to exist in the less common 3′-OH form, either by itself or together with the 3′-P form (Table 1). Interestingly, the 2′, 3′-cP products were found to distribute disproportionally between fully digested and miscleaved oligonucleotide populations, accounting for 13.2% ( 7/53) of the former and 43.5% ( 27/62) of the latter, respectively. This “enrichment” of digestion intermediates (e.g., 2′, 3′-cP) in miscleaved oligonucleotides is not surprising, and likely pointed to local regions of the mRNA sequence that were less efficiently digested by RNase T1 (e.g., due to secondary structures).

TABLE 1
RNase T1 digestion product of EGFP mRNA from in-solution complete digestion.

| # of | | | | | | | 2′, 3′-cP & |
|---|---|---|---|---|---|---|---|
| Missed | Unique | | 2′, 3′-cP | | 3′-P & | | 3′-P & 3′- |
| Cleavage | Sequences | 2′, 3′-CP | & 3′-P | 3′-P | 3′-OH | 3′-OH | OH |
| 0 | 52.7 ± 0.6 | 0.0 ± 0.0 | 7.0 ± 2.6 | 36.0 ± 3.0 | 8.7 ± 2.1 | 0.3 | 0.7 |
| 1 | 26.7 ± 3.2 | 16.3 ± 1.5 | 0.7 ± 0.6 | 9.0 ± 2.5 | — | 0.3 | — |
| 2 | 22.0 ± 1.0 | 4.0 ± 1.0 | 1.0 ± 0.0 | 15.0 ± 2.6 | — | 2 ± 1.0 | — |
| 3 | 9.3 ± 4.0 | 3.3 ± 2.1 | 0.0 ± 0.0 | 6.0 ± 2.0 | — | — | — |
| 4 | 3.7 ± 0.6 | 0.7 ± 0.6 | 1.0 ± 0.0 | 1.3 ± 6.0 | — | 0.7 | — |
| Total | 114.3 ± 3.1 | 24.3 ± 0.6 | 9.7 ± 3.1 | 67.7 ± 5.7 | 8.7 ± 2.1 | 3.3 ± 1.0 | 0.7 |

In contrast, FT-based limited digestion of EGFP mRNA generated predominantly homogeneous digestion products mainly composed of 2′, 3′-cP species (FIG. 12B and FIG. 18C). Specifically, 69.7% (182/261) of the identified unique oligonucleotides were found exclusively in the 2′, 3′-cP form, 25.3% ( 66/261) existed in both 2′, 3′-cP and 3′-P forms, and the remaining 5.0% ( 13/261) mostly adopted the 3′-P form (Table 2). Interestingly, among the 67 oligonucleotides that were associated with multiple 3′ structures, 50 were from fully digested products and only 17 were from miscleavage-containing products, each representing 92.5% ( 50/54) and 8.2% ( 17/207) of their corresponding populations, respectively.

TABLE 2
RNase T1 digestion product of EGFP mRNA from FT-based limited digestion.

| # of | | | | | | | 2′, 3′-cP & |
|---|---|---|---|---|---|---|---|
| Missed | Unique | | 2′, 3′-CP | | 3′-P & | | 3′-P & 3′- |
| Cleavage | Sequences | 2′, 3′-CP | & 3′-P | 3′-P | 3′-OH | 3′-OH | OH |
| 0 | 53.7 ± 0.6 | 1.7 ± 0.6 | 47.7 ± 2.5 | 3.0 ± 1.0 | — | — | 1.3 |
| 1 | 58.3 ± 4.0 | 49.3 ± 5.0 | 4.3 ± 1.2 | 4.3 ± 1.2 | — | 0.3 | — |
| 2 | 52.0 ± 1.0 | 45.7 ± 1.2 | 4.3 ± 1.2 | 4.3 ± 1.2 | — | — | — |
| 3 | 57.3 ± 0.6 | 49.3 ± 2.5 | 6.3 ± 1.5 | 6.3 ± 1.5 | — | — | — |
| 4 | 39.3 ± 5.1 | 35.7 ± 4.0 | 3.3 ± 1.5 | 3.3 ± 1.5 | — | 0.3 | — |
| Total | 260.7 ± 3.5 | 181.7 ± 7.2 | 66.0 ± 1.0 | 66.0 ± 1.0 | — | 0.7 | 1.3 |

The generation of a homogeneous 3′ structure in miscleaved oligonucleotides is highly desirable, as it enhances the detectability of the oligonucleotides by consolidating the MS signal. This is particularly beneficial in the context of limited digestion, where the abundance of individual oligonucleotide is usually reduced due to generation of multiple fragments with overlapping sequences. Notably, the favorable generation of 2′, 3′-cP over 3′-P products by FT-based limited digestion aligns well with the catalytic mechanism of RNase T1, which renders rapid generation of 2′,3′-cP intermediate species but slower subsequent hydrolysis reaction to form 3′-P products. Specifically, the 2′,3′-cP intermediates likely failed to undergo further hydrolysis to form 3′-P products, due to extremely short reaction time (30 s) allowed under the optimized FT-based digestion conditions. Together, these results highlight the advantage of FT-based limited digestion method in generating homogenous 2′,3′-cP products and their crucial role in improving overall sequence coverage for mapping mRNAs.

### Example 3. Characterization of Modified and Unmodified mRNAs by FT-Based Limited RNaseT1 Digestion

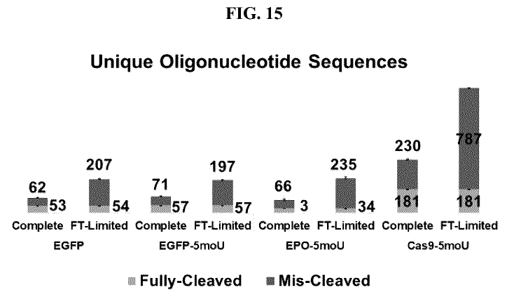

Synthetic mRNA with modified uridine has shown great potential for therapeutic applications, as the modifications can improve mRNA stability and reduce immunogenicity. To evaluate whether FT-based limited digestion can discriminate between unmodified mRNAs and those with chemical modifications, a modified EGFP mRNA, in which the uridine was substituted with 5-methoxy uridine (EGFP-5moU), was analyzed and compared with unmodified EGFP mRNA. The digestion performance was compared by assessing the number of unique oligonucleotides and the overall achieved mRNA sequence coverage. As shown in Table 3 and FIG. 15, both EGFP and EGFP-5moU mRNAs digested by FT-based limited RNase1 produced comparable numbers of unique oligonucleotides (261±4 and 254±6 from triplicate analyses, respectively). In particular, these unique oligonucleotides from both sample digests were predominantly composed of miscleavage-containing products (207±3 and 197±5, respectively), which aligned well with the expected outcome from the limited digestion approach. As a result, similar sequence coverages were achieved for EGFP-5moU mRNA (98.3%±0.3%) and unmodified EGFP mRNA (96.5%±1.1%).

TABLE 3
Number of miscleaved (unique) oligonucleotides and sequence
coverage of EGFP (720 nt) and EGFP-5moU (720 nt) using
FT-based limited RNase T1 digestion approach.

| | Fully-Cleaved | Mis-Cleaved | |
|---|---|---|---|
| | Oligo Sequences | Oligo Sequences | Sequence Coverage |
| EGFP (720 nt) | 54 ± 1 | 207 ± 3 | 96.5% ± 1.1% |
| EGFP-5moU | 57 ± 1 | 197 ± 5 | 98.3% ± 0.3% |
| (720 nt) | | | |

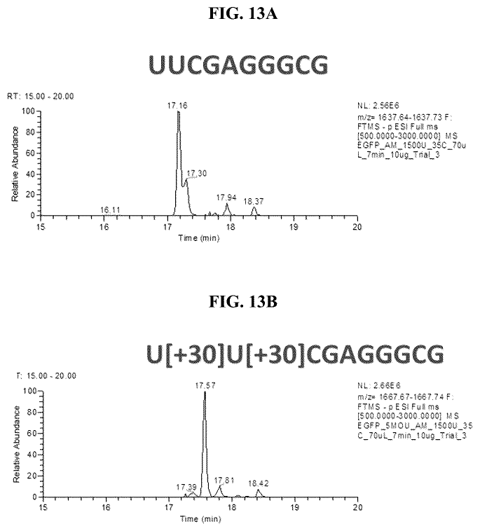

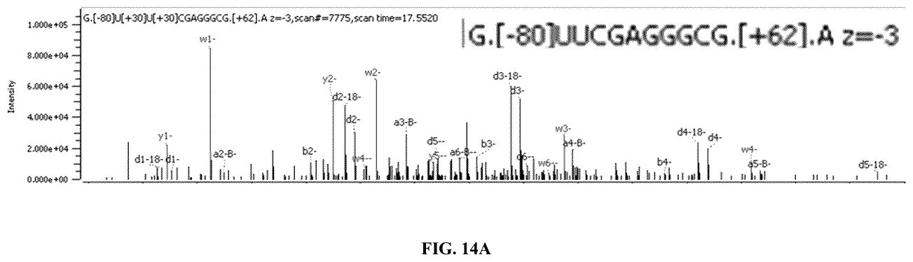

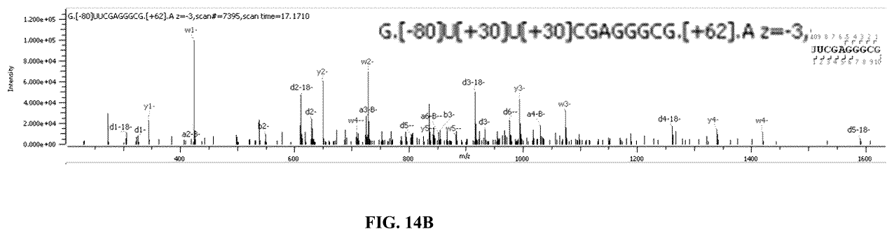

An exemplary miscleaved oligonucleotide (UUCGAGGGCG), with and without the 5moU modification, was further analyzed to assess digestion performance, LC separation, and MS/MS detection (FIGS. 13A, 13B, and 14). Specifically, the extracted ion chromatograms (XICs) displayed nearly identical intensities of both the unmodified and 5moU modified oligonucleotides from the corresponding digests (2.5-2.7E6, FIGS. 13A and 13B), suggesting a similar digestion efficiency for both samples. Under IP RPLC separation, the 5moU modified oligonucleotide eluted slightly later than the unmodified, presumably due to the increased hydrophobicity from the 5′-methoxyl group. Further, MS/MS analysis of the oligonucleotide was unaffected by the uridine modification and produced identical fragmentation pattern to that of the unmodified sequence (FIGS. 14A and 14B). Together, these results demonstrate that the FT-based RNase T1 digestion method can be generically applied to chemically modified mRNAs.

In addition to its utility for chemically modified mRNAs, the general applicability of a sequence mapping method across different mRNA molecules is equally important. Considering mRNAs are built by repetitions of four nucleotides and are largely “unstructured,” it is reasonable to hypothesize that the same limited digestion method could produce similar digestion outcomes for all mRNAs regardless of size and identity. To put this hypothesis to test, two additional mRNAs, Epo-5moU (582 nt) and Cas9-5moU (4245 nt), each representing a short or long sequence, were analyzed together with EGFP mRNA (720 nt) using the same FT-based limited RNase T1 digestion method. Specifically, in addition to the previously optimized RNase T1 amount, digestion temperature, and sample flow rate, the mRNA input (100 μL at 0.2 μg/μL) of each sample was also kept consistent. Further, solution-based complete digestion of these mRNAs was also conducted to enable comparison in sequence mapping performance.

The resulting quantitative comparison of the identified unique oligonucleotides and the calculated sequence coverages of each mRNA from both digestion methods are summarized in Table 4 and FIG. 15. As expected, the FT-based limited digestion method consistently delivered significantly higher sequence coverages for all three mRNAs (93.2%-98.3%), compared to those from the solution-based complete digestion method (61.5%-83.8%) (Table 4). Additionally, an apparent increase in the number of identified unique oligonucleotides, almost exclusively attributed to those with miscleavages, were consistently observed for all mRNAs by the FT-based digestion method compared to the solution-based digestion method (FIG. 15).

TABLE 4
Sequence coverage of EGFP (720 nt), EGFP-5moU (720 nt), Epo-5moU (582 nt) and
Cas9-5mouU (4245 nt) using FT-based limited RNase T1 digestion approach and
in-solution complete digestion method.
mRNA Sequence Coverage

| | EGFP | EGFP-5moU | Epo-5moU | Cas9-5moU |
|---|---|---|---|---|
| | (720 nt) | (720 nt) | (582 nt) | (4245 nt) |
| In solution | 79.8% ± 2.2% | 83.8% ± 2.3% | 80.2% ± 1.6% | 61.5% ± 2.5% |
| Complete Digestion | | | | |
| FT-based Limited | 96.5% ± 1.1% | 98.3% ± 0.3% | 96.9% ± 0.9% | 93.2% ± 0.9% |
| Digestion | | | | |

Interestingly, the obtained sequence coverages of mRNAs by solution-based digestion method appeared to be highly dependent on the mRNA length, which displayed a dramatic decrease of sequence coverage to 61.5% for Cas9 mRNA (4245 nt). This decrease in sequence coverage can be explained by the disproportionately large increase in mRNA lengths (6× increase from EGFP to Cas9 mRNA) compared to the relatively small increase in unique oligonucleotides generated by the solution-based digestion method (114 for EGFP and 410 for Cas9 mRNA), due to the highly repetitive nature of mRNA sequences. On the contrary, FT-based digestion effectively overcame this limitation by producing a large number of unique miscleaved oligonucleotides. For example, while both digestion methods yielded a comparable number of fully digested unique oligonucleotides from Cas9 mRNA (n=181), FT-based digestion produced significantly more miscleaved oligonucleotides (n=787 vs n=230, FIG. 15), resulting in an impressive increase in sequence coverage of Cas9 mRNA from 61.5% to 93.2%. The consistently high sequence coverage achieved for mRNAs ranging from 582 nt to 4245 nt in length further highlights the excellent versatility of the developed FT-based digestion method, making it suitable for characterizing a variety of therapeutic mRNAs.

### Example 4. Reproducibility of FT-Based Limited RNase T1 Digestion

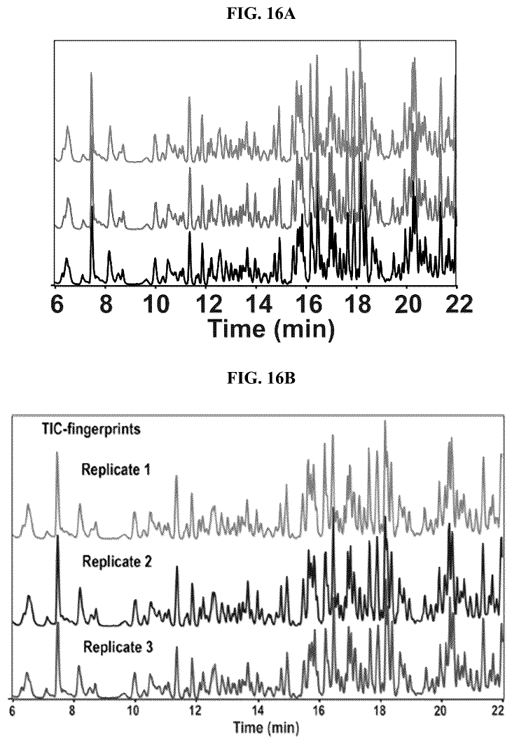

Reproducibility is an important consideration when developing analytical methods for routine application. To evaluate if the developed FT-based limited RNase T1 digestion method can robustly generate miscleaved oligonucleotides with reproducible patterns, three replicate digestions of EGFP mRNA were conducted and the final products were analyzed by LC-MS/MS. The results show that similar TIC profiles were obtained across the three replicates (FIGS. 16A and 16B). The peak features and retention times of the individual peaks were highly consistent among the triplicate analyses, suggesting that highly comparable digestion products were produced.

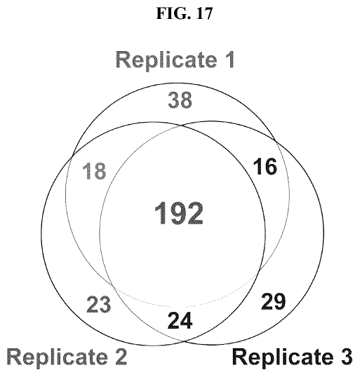

Further, the identified oligonucleotides from each analysis were compared and grouped by their occurrence in different replicates. As shown in FIG. 17, the identified oligonucleotides exhibited high reproducibility across the triplicate analyses, with a total of 192 oligonucleotides, which accounted for more than 73% of the entire population, consistently identified in all replicates. Although a small number of oligonucleotides were uniquely identified in each replicate, they were often present at low abundances, which led to inconsistent identification by data dependent MS2 analysis (e.g., due to exclusion from MS2 or poor MS2 quality).

Nevertheless, thanks to the presence of other redundant oligonucleotides with overlapping sequences, they did not impact the overall sequence coverage. Indeed, similar sequence coverages of EGFP mRNA were also achieved from the triplicate analyses, with an average of 96.5% (±1.2%). Together, these results indicate that missed cleavages were not generated randomly under the FT-based limited RNase T1 digestion conditions. Instead, these results indicate that the enzyme preferentially cleaves more accessible regions within the mRNA sequence and only cleaves less accessible regions (e.g., with secondary structures) in a limited fashion. It is hence concluded that the pattern of oligonucleotides produced by this FT-based limited digestion approach is reproducible and can be implemented for reliable mRNA sequence mapping. Additionally, with the multiplexing capability from the AssayMAP platform, this workflow can be implemented in a high-throughput manner, allowing for the digestion of up to 96 samples simultaneously.

Endoribonuclease digestion coupled with LC-MS/MS characterization is an attractive approach for mRNA sequence mapping. Traditional in-solution digestion methods using RNase T1 often generate short and non-unique oligonucleotide fragments that cannot be mapped to the mRNA sequence and, therefore, are limited in achieving sufficient sequence coverages. An FT-based limited RNase T1 digestion method in combination with LC-MS/MS was developed to enable high-sequence coverage mapping of therapeutic mRNAs. The workflow was built on an AssayMAP Bravo automation platform that enables precise control of digestion conditions. Using EGFP mRNA as a model system, the FT-based digestion approach was optimized to generate a high abundance of miscleavage-containing oligonucleotides with unique sequences, achieving an unprecedentedly high sequence coverage (>95% for EGFP mRNA). In particular, the FT-based limited digestion method, unlike the conventional in-solution digestion method, produced primarily homogeneous 2′,3′-cyclic-phosphorylated oligonucleotide products, which is essential for improving MS detection sensitivity. Further, the FT-based limited digestion method can be universally applied to chemically modified mRNAs and mRNAs of different lengths (up to 4245 nt tested) and consistently achieve high sequence coverage (>93%). Additionally, because the method was executed on an automation platform, the method was highly reproducible and provided opportunities for high-throughput analysis. Together, these features make this FT-based limited digestion method highly desirable during the development of therapeutic mRNAs, allowing for routine applications in industrial laboratories.

All references cited herein, including U.S. patent and applications are incorporated by reference in their entirety. The present disclosure is not to be limited in terms to the particular examples described in this application, which are intended as illustrations of various aspects. Many modifications and variations can be made without departing from its spirit and scope, as will be apparent to those skilled in the art. Functionally equivalent methods within the scope of the disclosure, in addition to those enumerated herein, will be apparent to those skilled in the art from the foregoing descriptions. Such modifications and variations are intended to fall within the scope of the appended claims.

### EMBODIMENTS

The following list of embodiments is intended to complement, rather than displace or supersede, the previous descriptions.

### Embodiments Set I

Embodiment I-1. A method of identifying and/or characterizing a polynucleotide of interest in a sample, comprising:
incubating a digestive enzyme with a linker to form a linked digestive enzyme;
contacting the linked digestive enzyme to a solid support within a cartridge to form an immobilized digestive enzyme;
contacting the sample containing the polynucleotide of interest to the immobilized digestive enzyme within the cartridge to form a digested sample;
eluting the digested sample to form an eluate; and
subjecting the eluate containing the digested sample to liquid chromatography-mass spectrometry (LC-MS) analysis to identify and/or characterize the at least one polynucleotide of interest.

Embodiment I-2. The method of embodiment 1, wherein said digestive enzyme is RNase T1.

Embodiment I-3. The method of any of the preceding embodiments, wherein said linker is biotin.

Embodiment I-4. The method of any of the preceding embodiments, wherein said solid support is streptavidin.

Embodiment I-5. The method of any of the preceding embodiments, wherein said cartridge is an AssayMap cartridge.

Embodiment I-6. The method of any of the preceding embodiments, wherein said cartridge contains a 5 μL resin bed.

Embodiment I-7. The method of any of the preceding embodiments, wherein the digestion is carried out using an AssayMAP.

Embodiment I-8. The method of any of the preceding embodiments, wherein the amount of linked digestive enzyme is about 500 units to about 3000 units.

Embodiment I-9. The method of any of the preceding embodiments, wherein the amount of linked digestive enzyme is about 1500 units.

Embodiment I-10. The method of any of the preceding embodiments, wherein the digestion is carried out at a temperature of about 25° C. to about 45° C.

Embodiment I-11. The method of any of the preceding embodiments, wherein the temperature is about 35° C.

Embodiment I-12. The method of any of the preceding embodiments, wherein the digestion is carried out for about 5 minutes to about 15 minutes.

Embodiment I-13. The method of any of the preceding embodiments, wherein the digestion is carried out for about 10 minutes.

Embodiment I-14. The method of any of the preceding embodiments, wherein said liquid chromatography is ion pairing reverse phase liquid chromatography.

Embodiment I-15. The method of any of the preceding embodiments, wherein said mass spectrometry is performed in tandem.

Embodiment I-16. The method of any of the preceding embodiments, wherein the digestion is a flow-through digestion.

### Embodiments Set II

Embodiment II-1. A method for mapping the sequence of a polynucleotide of interest in a sample, comprising:
immobilizing a digestive enzyme to a solid surface;
contacting a sample including the polynucleotide of interest to the solid surface in flow-through mode, wherein the digestive enzyme immobilized on the solid surface digests the polynucleotide of interest
collecting a flow-through fraction comprising the digested polynucleotide of interest; and
subjecting the flow-through to liquid chromatography-mass spectrometry analysis to map the sequence of the polynucleotide of interest.

Embodiment II-2. The method of embodiment 1, wherein the polynucleotide of interest is a mRNA.

Embodiment II-3. The method of any of the preceding embodiments, wherein the length of the mRNA is about 700 nt to about 5000 nt.

Embodiment II-4. The method of any of the preceding embodiments, wherein the mRNA comprises a modified nucleoside.

Embodiment II-5. The method of any of the preceding embodiments, wherein the nucleoside is uridine.

Embodiment II-6. The method of any of the preceding embodiments, wherein the modified nucleoside comprises pseudouridine (ψ), N1-methylpseudouridine (m1ψ), or 5-methoxyuridine (5moU).

Embodiment II-7. The method of any of the preceding embodiments, wherein the amount of polynucleotide of interest in the sample is about 20 μg.

Embodiment II-8. The method of any of the preceding embodiments, wherein the digestive enzyme is RNase T1.

Embodiment II-9. The method of any of the preceding embodiments, wherein immobilizing, contacting, and collecting are automated.

Embodiment II-10. The method of any of the preceding embodiments, wherein immobilizing comprises contacting a biotinylated digestive enzyme to a solid surface that is coated with avidin, streptavidin, or a variant thereof.

Embodiment II-11. The method of any of the preceding embodiments, wherein the solid surface is a microcartridge.

Embodiment II-12. The method of any of the preceding embodiments, wherein the amount of immobilized digestive enzyme is about 500 units to about 3000 units.

Embodiment II-13. The method of any of the preceding embodiments, wherein the amount of immobilized digestive enzyme is about 1500 units.

Embodiment II-14. The method of any of the preceding embodiments, wherein the sample is contacted to the solid surface at a temperature of about 25° C. to about 45° C.

Embodiment II-15. The method of any of the preceding embodiments, wherein the temperature is about 35° C.

Embodiment II-16. The method of any of the preceding embodiments, wherein the sample is contacted to the solid surface at a flow rate of about 10 μL/min to about 20 μL/min.

Embodiment II-17. The method of any of the preceding embodiments, wherein the flow rate is about 10 μL/min.

Embodiment II-18. The method of any of the preceding embodiments, wherein the total time the sample is contacted to the solid surface is about 5 to 15 minutes.

Embodiment II-19. The method of any of the preceding embodiments, wherein the total time is about 10 minutes.

Embodiment II-20. The method of any of the preceding embodiments, wherein the liquid chromatography is ion-pairing reversed phase liquid chromatography.

Embodiment II-21. The method of any of the preceding embodiments, wherein the mass spectrometry is performed in tandem.

Embodiment II-22. The method of any of the preceding embodiments, wherein the mass spectrometer is an electrospray ionization mass spectrometer, nano-electrospray ionization mass spectrometer, quadropole mass spectrometer, an Orbitrap-based mass spectrometer, or a hybrid quadropole-Orbitrap-based mass spectrometer,

Embodiment II-23. The method of any of the preceding embodiments, wherein the mass spectrometer is coupled to the liquid chromatography system.

Embodiment II-24. The method of any of the preceding embodiments, wherein a splitter is used to couple the mass spectrometer to the liquid chromatography system.

Embodiment II-25. The method of any of the preceding embodiments, wherein the liquid chromatography system is further coupled to an ultraviolet detector.

Embodiment II-26. The method of any of the preceding embodiments, wherein the UV detector monitors absorbance at a wavelength of 260 nm.

Embodiments set III.

Embodiment III-1. A method for identifying and/or characterizing the sequence of a polynucleotide of interest in a sample, comprising:
contacting the sample comprising the polynucleotide of interest with a solid support having a digestive enzyme immobilized thereto to thereby form a digested sample;
eluting the digested sample from the solid support to form an eluate; and
analyzing the eluate by liquid chromatography-mass spectrometry, to identify and/or characterize the sequence of the polynucleotide of interest.

Embodiment III-2. The method of embodiment 1, wherein the polynucleotide of interest is a mRNA.

Embodiment III-3. The method of any of the preceding embodiments, wherein the length of the mRNA is about 700 nucleotides to about 5000 nucleotides.

Embodiment III-4. The method of any of the preceding embodiments, wherein the mRNA comprises a modified uridine.

Embodiment III-5. The method of any of the preceding embodiments, wherein the modified uridine comprises pseudouridine (ψ), N1-methylpseudouridine (m1ψ), or 5-methoxyuridine (5moU).

Embodiment III-6. The method of any of the preceding embodiments, wherein the amount of polynucleotide of interest in the sample is about 20 μg.

Embodiment III-7. The method of any of the preceding embodiments, wherein the digestive enzyme is RNase T1.

Embodiment III-8. The method of any of the preceding embodiments, wherein the solid support is a microcartridge.

Embodiment III-9. The method of any of the preceding embodiments, wherein the amount of immobilized digestive enzyme is about 500 units to about 3000 units.

Embodiment III-10. The method of any of the preceding embodiments, wherein the sample is contacted to the solid support at a temperature of about 25° C. to about 45° C.

Embodiment III-11. The method of any of the preceding embodiments, wherein the sample is contacted to the solid support at a flow rate of about 10 μL/min to about 20 μL/min.

Embodiment III-12. The method of any of the preceding embodiments, wherein the total reaction time the sample is contacted to the solid support is about 5 to 15 minutes.

Embodiment III-13. The method of any of the preceding embodiments, wherein the eluting is performed for about 15 to about 60 seconds.

Embodiment III-14. The method of any of the preceding embodiments, wherein the liquid chromatography is ion-pairing reversed phase liquid chromatography.

Embodiment III-15. The method of any of the preceding embodiments, wherein the mass spectrometer is coupled to the liquid chromatography system.

Embodiment III-16. The method of any of the preceding embodiments, wherein the liquid chromatography system is further coupled to an ultraviolet detector.

Embodiment III-17. The method of any of the preceding embodiments, wherein the ultraviolet detector monitors absorbance at a wavelength of 260 nm.

Embodiment III-18. The method of any of the preceding embodiments, wherein the identifying and/or characterizing comprises mapping the digested polynucleotide to an undigested polynucleotide.

Embodiment III-19. The method of any of the preceding embodiments, comprising applying the sample to the solid support such that the sample flows through the solid support and forms the digested sample.

What is claimed is:

1. A method for identifying and/or characterizing the sequence of a polynucleotide of interest in a sample, comprising:
contacting the sample comprising the polynucleotide of interest with a solid support having a digestive enzyme immobilized thereto to thereby form a digested sample;
eluting the digested sample from the solid support to form an eluate; and
analyzing the eluate by liquid chromatography-mass spectrometry, to identify and/or characterize the sequence of the polynucleotide of interest.

2. The method of claim 1, wherein the polynucleotide of interest is a mRNA.

3. The method of claim 2, wherein the length of the mRNA is about 700 nucleotides to about 5000 nucleotides.

4. The method of claim 2, wherein the mRNA comprises a modified uridine.

5. The method of claim 4, wherein the modified uridine comprises pseudouridine (ψ), N1-methylpseudouridine (m1ψ), or 5-methoxyuridine (5moU).

6. The method of claim 1, wherein the amount of polynucleotide of interest in the sample is about 20 μg.

7. The method of claim 1, wherein the digestive enzyme is RNase T1.

8. The method of claim 1, wherein the solid support is a microcartridge.

9. The method of claim 1, wherein the amount of immobilized digestive enzyme is about 500 units to about 3000 units.

10. The method of claim 1, wherein the sample is contacted to the solid support at a temperature of about 25° C. to about 45° C.

11. The method of claim 1, wherein the sample is contacted to the solid support at a flow rate of about 7 μL/min to about 20 μL/min.

12. The method of claim 1, wherein the total reaction time the sample is contacted to the solid support is about 5 to 15 minutes.

13. The method of claim 1, wherein the eluting is performed for about 15 to about 60 seconds.

14. The method of claim 1, wherein the liquid chromatography is ion-pairing reversed phase liquid chromatography.

15. The method of claim 1, wherein the mass spectrometer is coupled to the liquid chromatography system.

16. The method of claim 1, wherein the liquid chromatography system is further coupled to an ultraviolet detector.

17. The method of claim 16, wherein the ultraviolet detector monitors absorbance at a wavelength of 260 nm.

18. The method of claim 1, wherein the identifying and/or characterizing comprises mapping the digested polynucleotide to an undigested polynucleotide.

19. The method of claim 1, comprising applying the sample to the solid support such that the sample flows through the solid support and forms the digested sample.
